(19) United States
(12) Patent Application Publication
(10) Pub. No.: US 2025/0003847 A1
(43) Pub. Date: Jan. 2, 2025

(54) INTEGRATED MACHINE FOR FULLY AUTOMATIC CORE DRILLING AND DIRECT PULLING AND A DETECTION METHOD THEREOF

(71) Applicant: Hunan Academy of Building Research Co., Ltd, Changsha (CN)

(72) Inventors: Wenming Wang, Changsha (CN); Yongjun Dai, Changsha (CN); Lihua Zhou, Changsha (CN); Hu Yu, Changsha (CN); Jianjun Tian, Changsha (CN); Xianghua Zhou, Changsha (CN); Peiqing Zhao, Changsha (CN); Qinglu Xiao, Changsha (CN); Baohong Wang, Changsha (CN); Huasheng Yin, Changsha (CN); Wen Su, Changsha (CN); Dabiao Zhu, Changsha (CN)

(21) Appl. No.: 18/609,474

(22) Filed: Mar. 19, 2024

(30) Foreign Application Priority Data
Jun. 27, 2023 (CN) 202310764571.1

(51) Int. Cl. G01N 3/16; G01N 3/04; G01N 33/38

(52) U.S. Cl. CPC G01N 3/16; G01N 3/04; G01N 33/383; G01N 2203/0017; G01N 2203/0067; G01N 2203/0244

(57) ABSTRACT

An integrated machine for fully automatic core drilling and direct pulling is disclosed, including a housing, a support member, a pulling mechanism, and a pulling joint. The pulling joint is configured to hold a top of a test piece for pulling testing. The pulling mechanism includes a worm gear motor, a drive screw set at an output end of the worm gear motor, a drive support rod, a tension sensor set at the other end of the drive support rod, an adjustable link block at one end connected to the tension sensor, and a pulling screw connected to the other end of the adjustable link block. The integrated machine has a simple structure, lightweight equipment, which can be handheld and easy to operate, thus do not need to have operating experience, thereby realizing core drilling and direct pulling test in one.

### TECHNICAL FIELD

The present application relates to the technical field of concrete compressive strength testing, and in particular to an integrated machine for fully automatic core drilling and direct pulling and a detection method thereof.

### BACKGROUND

Direct pulling method is a technology used to test strength of concrete, which is a new type of non-destructive testing technology for concrete developed by engineering quality inspectors of our country on the basis of summarizing experiences of decades of non-destructive testing technologies, with features of small damage to structure, high precision and wide test range (suitable for concrete compressive strength testing within 10 MPa˜100 MPa concrete compressive strength).

In existing technologies, technology principle of the direct pulling method for testing concrete strength is that, a metal direct pulling joint on a direct pulling instrument is bonded to a concrete tested piece by a binder (generally epoxy resin), and when the strength of the binder reaches a certain degree, a concrete core sample in the concrete tested piece is pulled out with the metal direct pulling joint, and then the concrete compressive strength of the concrete tested piece is presumed according to a measured pulling strength. In such operation, it need to drill core for sampling on the concrete tested piece, and then tested. However, in the existing technologies, use of the direct pulling method to test concrete strength being of cumbersome technical operation steps, long operating time, with large equipment so that it is inconvenient to carry on-site, and its detection process is complex and also subject to operations of a operator's operating factors, resulting in technical problems such as detection of lower precision and not environmental friendly issues.

### SUMMARY

In view of defects in the existing technologies, the present disclosure aims to provide an integrated machine for fully automatic core drilling and direct pulling to address problems mentioned in the above background.

In some embodiments of the present disclosure, an integrated machine for fully automatic core drilling and direct pulling is provided, including a housing, a support member disposed at bottom of the housing, a pulling mechanism disposed inside the housing, and a pulling joint disposed below the housing in connection with the pulling mechanism. Herein the pulling joint is configured to clamp a top of a test piece for pulling testing.

The support member is configured to be disposed around the pulling joint.

The pulling mechanism includes a worm gear motor set in a lower part of the housing, a drive screw set at an output end of the worm gear motor, a drive support rod fixedly connected to the drive screw, a tension sensor set at the other end of the drive support rod, an adjustable link block at one end connected to the tension sensor, a pulling screw connected to the other end of the adjustable link block, a power supply and a controller set inside the housing, and a switch and a touch screen set at a top of the housing.

The adjustable link block is configured to adjust a distance between the tension sensor and the pulling screw.

One end of the pulling screw extends out of the housing, and the pulling joint is connected to the one end of the pulling screw extending out of the housing.

The tension sensor is configured to measure a tension data when the pulling joint direct pulls out of the test piece to break.

The integrated machine for fully automatic core drilling and direct pulling further includes a core drilling motor disposed at an upper end of the pulling screw, and a drill bit removably connected to a lower end of the pulling screw.

The controller is electrically connected to the power supply, the switch, the touch screen, the worm gear motor, the tension sensor, and the core drilling motor.

Preferably, the pulling joint includes a limit member and a clamping member those connected to the pulling screw, and an ejector spring disposed between the limit member and the clamping member, one end of the limit member is configured with a concave cavity with an opening, an inner wall of the concave cavity is tapered to a certain degree, the clamping member is disposed in the concave cavity, the clamping member comprises a plurality of clamping jaws symmetrically disposed along a center axis of the limit member, outer walls of the clamping jaws are configured with abutting portions adapted to the concave cavity, the limit member is configured with a locking nut at its top and the locking nut is adapted to the pulling screw for limiting the limit member along a direction of pulling the pulling screw.

Preferably, a top of the limit member is configured with a planar sliding bearing and the planar sliding bearing is sleeved on the pulling screw.

Preferably, the worm gear motor is configured with two and such two worm gear motors are disposed opposite to each other in the lower part of the housing, and two drive screws are set diagonally along both sides of the pulling screw.

Preferably, the two drive screws are configured with sensor fixing blocks attached to their tops, and the tension sensor is disposed on the sensor fixing blocks.

Preferably, the clamping jaws are configured with anti-slip bumps on their inner side for anti-slip when the clamping jaws are clamping the test piece.

Preferably, the support member includes support posts arranged annularly around the pulling joint or support plates arranged annularly.

Preferably, handles are provided on both sides of an upper end of the housing.

In some other embodiments of the present disclosure, the present disclosure further provides a detection method applicable to the integrated machine for fully automatic core drilling and direct pulling described above, including following steps 1 to 4.

Step 1, a measured plane on a concrete matrix to be tested is selected.

Step 2, the drill bit is installed, the concrete matrix to be tested is core drilled to take the test piece, the pulling joint of a direct pulling instrument is used to hold the test piece, and the switch is activated for pulling.

Step 3, the test piece of the concrete matrix to be tested is subjected a tensile force until the concrete matrix to be tested is damaged by pulling and a concrete failure surface will be appeared at a certain position of the test piece of the concrete matrix to be tested.

Step 4, the direct pulling instrument records a peak value of the tensile force at a moment when the concrete matrix to be tested is damaged by pulling, the controller calculates through a set arithmetic program, and the touch screen is displayed a compressive strength of the test piece of the concrete matrix to be tested in real time.

The integrated machine for fully automatic core drilling and direct pulling of the present disclosure has at least following advantages and beneficial effects compared with the existing technologies. The integrated machine for fully automatic core drilling and direct pulling provided by the present disclosure, has a simple structure and lightweight equipment, which can be handheld to facilitate on-site carry. In addition, it is also more environmentally friendly, low cost, and easy to operate, no need to have operating experience, so as to be able to realize core drilling and direct pulling test in one, and directly display values of the compressive strength of the test piece of the concrete, test results are intuitive. Furthermore, no weights are required for calibration, and just enter sensor parameters to complete the calibration.

### BRIEF DESCRIPTION OF THE DRAWINGS

FIG. 1 is a three-dimensional structural schematic diagram of an integrated machine for fully automatic core drilling and direct pulling in accordance with an embodiment of the present disclosure.

FIG. 2 is a section schematic diagram of the integrated machine for fully automatic core drilling and direct pulling in accordance with an embodiment of the present disclosure.

FIG. 3 is a structural schematic diagram of a pulling joint in accordance with an embodiment of the present disclosure.

In the drawings, reference signs are as follows. 10. Housing, 20. Support member, 30. Pulling mechanism, 31. Worm gear motor, 32. Drive screw, 33. Drive support rod, 34. Tension sensor, 341. Sensor fixing block, 35. Adjustable link block, 36. Pulling screw, 37. Power supply, 38. Controller, 39. Touch screen, 40. Pulling joint, 41. Limit member, 42. Clamping member, 43. Ejector spring, 44. Concave cavity, 45. Abutting portion, 46. Locking nut, 47. Planar sliding bearing, 50. Drill bit, 60. Core drilling motor, 70. Switch, 80. Handle.

### DETAILED DESCRIPTION OF THE EMBODIMENTS

The present disclosure will be described in detail below in connection with specific embodiments. The following embodiments will help those skilled in the art to further understand the present invention, but do not limit the present invention in any way. It should be noted that, for a person of ordinary skill in the art, a number of deformations and improvements can be made without departing from the concept of the present invention, which are all within the scope of protection of the present invention.

In the description of the present disclosure, it is to be understood that these terms “center”, “longitudinal”, “lateral”, “length”, “width”, “thickness”, “up”, “down”, “front”, “back”, “left”, “right”, “vertical”, “horizontal”, “top”, “bottom”, “inside”, “outside”, “clockwise”, “counterclockwise”, and the like indicate orientation or positional relationships that are based on those shown in the accompanying drawings, solely for the purpose of facilitating the description of the present invention and simplifying the description, they are not intended to indicate or imply that a device or an element referred to must have a particular orientation and be constructed and operated in a particular orientation, therefore they are not to be construed as a limitation of the present invention.

In addition, the terms “first”, “second” are used for descriptive purposes only but are not to be understood as indicating or implying relative importance or implicitly specifying the number of technical features indicated. Thus, a feature defined with the terms “first” or “second” may expressly or impliedly include one or more such features. in the description of the present invention, “more than one” means two or more, unless otherwise expressly and specifically limited.

In the present disclosure, unless otherwise expressly specified and limited, the terms “installed”, “connected”, “connection”, “fixed”, etc. are to be understood in a broad sense. For example, it may be a fixed connection, a detachable connection, or a connection in one piece. It also may be a mechanical connection or an electrical connection, it still may be a direct connection or an indirect connection through an intermediate medium, and it yet still may be a connection within two elements. For those of ordinary skill in the art, a specific meaning of the above terms in the present invention may be understood based on the context of a specific situation.

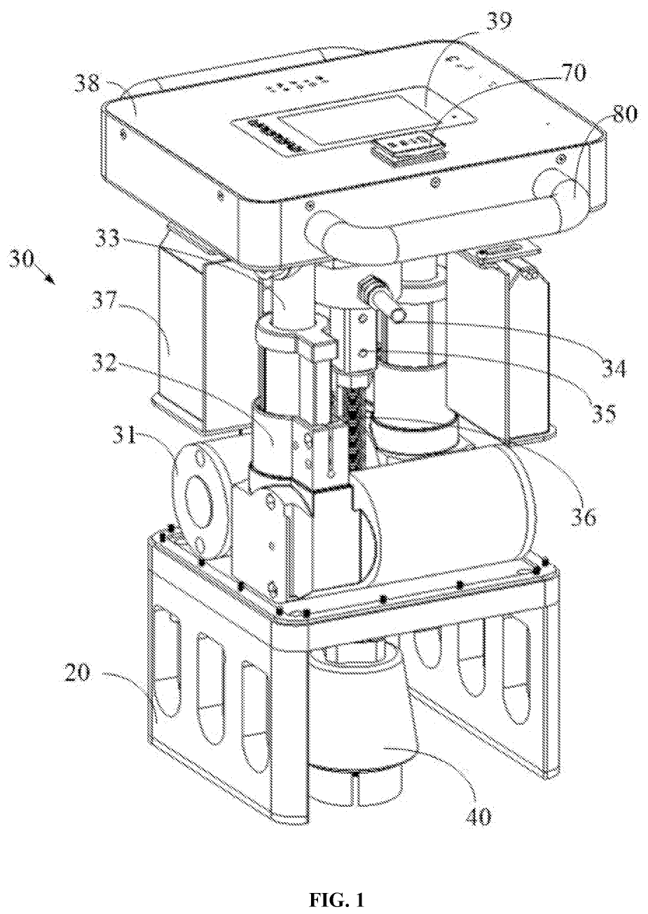

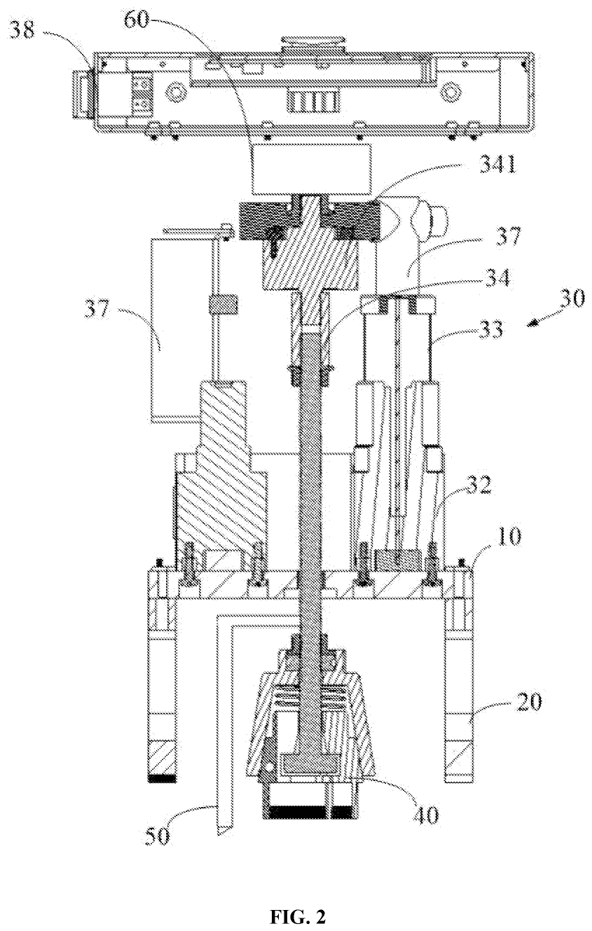

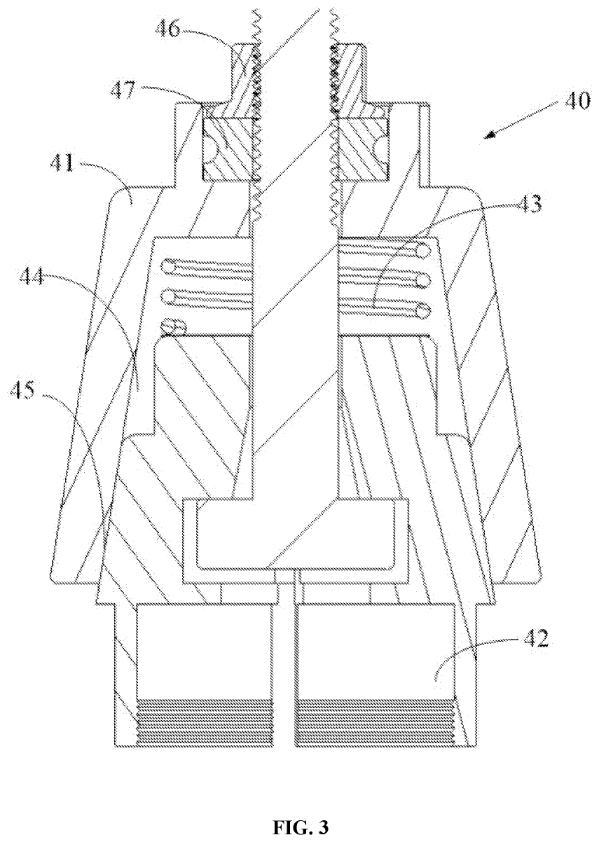

Referring to FIG. 1 to FIG. 3, which shows specific structures of an integrated machine for fully automatic core drilling and direct pulling in accordance with some embodiments of the present disclosure.

In some embodiments of the present disclosure, an integrated machine for fully automatic core drilling and direct pulling is provided, including a housing 10, a support member 20 disposed at bottom of the housing 10, a pulling mechanism 30 disposed inside the housing 10, and a pulling joint 40 disposed below the housing 10 in connection with the pulling mechanism 30. Herein the pulling joint 40 is configured to clamp a top of a test piece for pulling testing, and the support member 20 is configured to be disposed around the pulling joint 40. The pulling mechanism 30 includes a worm gear motor 31 set in a lower part of the housing 10, a drive screw 32 set at an output end of the worm gear motor 31, a drive support rod 33 fixedly connected to the drive screw 32, a tension sensor 34 set at the other end of the drive support rod 33, an adjustable link block 35 at one end connected to the tension sensor 34, a pulling screw 36 connected to the other end of the adjustable link block 35, a power supply 37 and a controller 38 set inside the housing 10, and a switch 70 and a touch screen 39 set at a top of the housing 10. The power supply 37 is a rechargeable lithium battery or a photovoltaic rechargeable battery, light sources are set on both sides of the drive screw 32, and the controller 38 is set on the top. The adjustable link block 35 is used to adjust a distance between the tension sensor 34 and the pulling screw 36. The adjustable link block 35 is provided with a plurality of connection holes linking the tension sensor 34 at equally spaced intervals. One end of the pulling screw 36 extends out of the housing 10, and the pulling joint 40 is connected to the one end of the pulling screw 36 extending out of the housing 10. The tension sensor 34 is used to measure a tension data when the pulling joint 40 direct pulls out of the test piece to break. Moreover, the integrated machine for fully automatic core drilling and direct pulling further includes a core drilling motor 60 disposed at an upper end of the pulling screw 36, and a drill bit 50 removably connected to a lower end of the pulling screw 36. The drill bit 50 includes a connecting portion connected to the pulling screw 36, the connecting portion inside is provided with a cavity to hold the pulling joint 40, the connecting portion at a lower end is provided with a core drill bit, and the core drill bit drills a desired annular groove around the test piece in the concrete matrix to be tested. The pulling joint 40 is inserted into the annular groove to clamp the test piece for pulling.

The controller 38 is electrically connected to the power supply 37, the switch 70, the touch screen 39, the worm gear motor 31, the tension sensor 34, and the core drilling motor 60. The controller 38 receives signals from the switch 70, the touch screen 39 and the tension sensor 34, which in turn drives the worm gear motor 31 and the core drilling motor 60 to work. In some other embodiments of the present disclosure, a Bluetooth module and a Type interface may also be provided for direct export of detected test data.

In some other embodiments of the present disclosure, the pulling joint 40 includes a limit member 41 and a clamping member 42 those connected to the pulling screw 36, and an ejector spring 43 disposed between the limit member 41 and the clamping member 42. One end of the limit member 41 is configured with a concave cavity 44 with an opening, an inner wall of the concave cavity 44 is tapered to a certain degree, the clamping member 42 is disposed in the concave cavity 44, the clamping member 42 includes a plurality of clamping jaws symmetrically disposed along a center axis of the limit member 41, outer walls of the clamping jaws are configured with abutting portions 45 adapted to the concave cavity 44, the limit member 41 is configured with a locking nut 46 at its top and the locking nut 46 is adapted to the pulling screw 36 for limiting the limit member 41 along a direction of pulling the pulling screw 36. The drive screw 32 pulls the pulling screw 36 upward, and the clamping member 42 moves upward under an action of the pulling screw 36, the abutting portions 45 at the outer wall of the clamping jaws and the inner wall of the limit member 41 abut to limit, and the limit member 41 is limited in an upward direction along a centerline of the pulling screw 36 under an action of the locking nut 46 to realize the clamping of the test piece. After the pulling joint 40 clamps the test piece, the pulling screw 36 pulls it upward until the tested concrete is damaged by pulling, and the sensor records a peak value of the pulling force at the moment of destruction of the tested concreted by pulling.

In some embodiments of the present disclosure, a top of the limit member 41 is configured with a planar sliding bearing 47, and the planar sliding bearing 47 is sleeved on the pulling screw 36 for facilitating a movement of the clamping member 42 to clamp the test piece.

In some embodiments of the present disclosure, the worm gear motor 31 is configured with two and such two worm gear motors 31 are disposed opposite to each other in the lower part of the housing 10, and two drive screws 32 are set diagonally along both sides of the pulling screw 36. In this way, it has a compact structure, the pulling screw 36 has a balanced force and is easy to operate.

In some embodiments of the present disclosure, the two drive screws 32 are configured with sensor fixing blocks 341 attached to their tops, and the tension sensor 34 is disposed on the sensor fixing blocks 341. The sensor fixing blocks 341 each further includes a limit chamber containing the tensile sensor 34 for preventing the pulling screw 36 from being damaged by torque on the tensile sensor 34 during rotation. The pulling screw 36 is provided with a limit projection, and the limit chamber is provided with a recess adapted to the limit projection.

In some embodiments of the present disclosure, the clamping jaws are configured with anti-slip bumps on their inner side to increase the friction between the clamping jaws and the test piece for anti-slip when the clamping jaws are clamping the test piece.

In some embodiments of the present disclosure, the support member 20 includes support posts arranged annularly around the pulling joint 40 or support plates arranged annularly.

In some embodiments of the present disclosure, handles 80 are provided on both sides of an upper end of the housing 10, which can facilitate a test to be carried out directly by hand.

In some other embodiments of the present disclosure, a detection method applicable to the integrated machine for fully automatic core drilling and direct pulling described above is provided, including following steps 1 to 4.

Step 1, a measured plane on a concrete matrix to be tested is selected.

Step 2, the drill bit is installed, the concrete matrix to be tested is core drilled to take the test piece, the pulling joint of a direct pulling instrument is used to hold the test piece, and the switch is activated for pulling.

Step 3, the test piece of the concrete matrix to be tested is subjected a tensile force until the concrete matrix to be tested is damaged by pulling and a concrete failure surface will be appeared at a certain position of the test piece of the concrete matrix to be tested.

Step 4, the direct pulling instrument records a peak value of the tensile force at a moment when the concrete matrix to be tested is damaged by pulling, the controller calculates through a set arithmetic program, and the touch screen is displayed a compressive strength of the test piece of the concrete matrix to be tested in real time. In one example, the strength measurement formula is

$${f}_{cu , i}^{c} = 21.049 {f}_{zbd , i}^{0.9402} .$$

The integrated machine for fully automatic core drilling and direct pulling provided by the present disclosure, has a simple structure and lightweight equipment, which can be handheld to facilitate on-site carry. In addition, it is also more environmentally friendly, low cost, and easy to operate, no need to have operating experience, so as to be able to realize core drilling and direct pulling test in one, and directly display values of the compressive strength of the test piece of the concrete, test results are intuitive. Furthermore, no weights are required for calibration, and just enter sensor parameters to complete the calibration.

The above is a further detailed description of the present invention in combination with specific embodiments, and it cannot be concluded that the specific embodiments of the present disclosure are limited to these descriptions only. For the person of ordinary skill in the art to which the present invention belongs, a number of simple deductions or substitutions may be made without departing from the concept of the present invention.

What is claimed is:

1. An integrated machine for fully automatic core drilling and direct pulling, comprising
a housing,
a support member disposed at bottom of the housing,
a pulling mechanism disposed inside the housing, and
a pulling joint disposed below the housing in connection with the pulling mechanism;
wherein the pulling joint is configured to clamp a top of a test piece for pulling testing, and the support member is configured to be disposed around the pulling joint;
wherein the pulling mechanism comprises a worm gear motor set in a lower part of the housing, a drive screw set at an output end of the worm gear motor, a drive support rod fixedly connected to the drive screw, a tension sensor set at the other end of the drive support rod, an adjustable link block at one end connected to the tension sensor, a pulling screw connected to the other end of the adjustable link block, a power supply and a controller set inside the housing, and a switch and a touch screen set at a top of the housing;
wherein the adjustable link block is configured to adjust a distance between the tension sensor and the pulling screw;
wherein one end of the pulling screw extends out of the housing, and the pulling joint is connected to the one end of the pulling screw extending out of the housing;
wherein the tension sensor is configured to measure a tension data when the pulling joint direct pulls out of the test piece to break; and
wherein the integrated machine for fully automatic core drilling and direct pulling further comprises a core drilling motor disposed at an upper end of the pulling screw, and a drill bit removably connected to a lower end of the pulling screw;
wherein the controller is electrically connected to the power supply, the switch, the touch screen, the worm gear motor, the tension sensor, and the core drilling motor; and
wherein the pulling joint comprises a limit member and a clamping member those connected to the pulling screw, and an ejector spring disposed between the limit member and the clamping member, one end of the limit member is configured with a concave cavity with an opening, an inner wall of the concave cavity is tapered to a certain degree, the clamping member is disposed in the concave cavity, the clamping member comprises a plurality of clamping jaws symmetrically disposed along a center axis of the limit member, outer walls of the clamping jaws are configured with abutting portions adapted to the concave cavity, the limit member is configured with a locking nut at its top and the locking nut is adapted to the pulling screw for limiting the limit member along a direction of pulling the pulling screw.

2. The integrated machine for fully automatic core drilling and direct pulling according to claim 1, wherein a top of the limit member is configured with a planar sliding bearing and the planar sliding bearing is sleeved on the pulling screw.

3. The integrated machine for fully automatic core drilling and direct pulling according to claim 1, wherein the worm gear motor is configured with two and such two worm gear motors are disposed opposite to each other in the lower part of the housing, and two drive screws are set diagonally along both sides of the pulling screw.

4. The integrated machine for fully automatic core drilling and direct pulling according to claim 1, wherein the two drive screws are configured with sensor fixing blocks attached to their tops, and the tension sensor is disposed on the sensor fixing blocks.

5. The integrated machine for fully automatic core drilling and direct pulling according to claim 1, wherein the clamping jaws are configured with anti-slip bumps on their inner side for anti-slip when the clamping jaws are clamping the test piece.

6. The integrated machine for fully automatic core drilling and direct pulling according to claim 1, wherein the support member comprises support posts arranged annularly around the pulling joint or support plates arranged annularly.

7. The integrated machine for fully automatic core drilling and direct pulling according to claim 1, wherein handles are provided on both sides of an upper end of the housing.

8. A detection method applicable to the an integrated machine for fully automatic core drilling and direct pulling of claim 1, comprising,
step 1, a measured plane on a concrete matrix to be tested is selected;
step 2, the drill bit is installed, the concrete matrix to be tested is core drilled to take the test piece, the pulling joint of a direct pulling instrument is used to hold the test piece, and the switch is activated for pulling;
step 3, the test piece of the concrete matrix to be tested is subjected a tensile force until the concrete matrix to be tested is damaged by pulling and a concrete failure surface will be appeared at a certain position of the test piece of the concrete matrix to be tested; and
step 4, the direct pulling instrument records a peak value of the tensile force at a moment when the concrete matrix to be tested is damaged by pulling, the controller calculates through a set arithmetic program, and the touch screen is displayed a compressive strength of the test piece of the concrete matrix to be tested in real time.
